(19) United States
(12) Patent Application Publication
(10) Pub. No.: US 2008/0319166 A1
(43) Pub. Date: Dec. 25, 2008

(54) Treatment of implantable medical devices resistant to calcification

(76) Inventor: Ming Shen, Malakoff (FR)

Correspondence Address: WENDEROTH, LIND & PONACK, L.L.P., 2033 K STREET N. W., SUITE 800, WASHINGTON, DC, 20006-1021, US

(21) Appl. No.: 12/213,338

(22) Filed: Jun. 18, 2008

(30) Foreign Application Priority Data
Jun. 20, 2007 (FR) 07 55890

(51) Int. Cl. C07K 1/107; C07K 14/75; C07K 14/78; C07K 14/745

(52) U.S. Cl. 530/345; 530/382; 530/380; 530/356; 530/353

(57) ABSTRACT

Treatment of implantable medical devices resistant to calcification The invention relates to a method for treating an implant comprising a protein-based substrate, including the following steps in which:

(A)—the protein-based substrate is treated with a compound containing at least one aldehyde group, then

(B)—the substrate is treated with a compound comprising a borohydride, then

(C)—the substrate resulting from step (B) is treated with a derivative containing a silane group.

The invention also relates to the treated protein-based implant obtained at the end of this method.

### TECHNICAL FIELD

The present invention relates to implantable medical devices (referred to hereinafter by the generic term “medical implant”). More precisely, the invention relates to protein-based medical implants, in particular collagen-based medical implants, rendered biocompatible and resistant to calcification, and more specifically to a method for preparing implants of this type.

### BACKGROUND TO THE INVENTION

Different types of medical implant exist, among which protein-based implants are included which are of particular benefit. These implants are particularly more advantageous than non-biological material-based implants in that they make it possible to avoid, inter alia, thromboses when they are inserted into a living organism, in particular into humans. However, in order to obtain such a biocompatibility, protein-based implants must be pre-treated.

The proteins present in the protein-based implants are, in fact, generally fibrous proteins (typically collagens) which comprise free amine functions. Said free amine functions are partly responsible for the implant being rejected in a living body, subject to the immune system recognising said free amine functions of the protein.

In order to avoid this occurrence of rejection, it is known to treat the implant with a difunctional aldehyde compound, typically glutaraldehyde.

The main aim of said difunctional aldehyde compound is to mask the amine functions. In this regard, the aldehyde functions react with the amine functions of the implant by forming imine functions (—C═N—). Furthermore, the use of a difunctional compound comprising two aldehyde functions allows the different protein fibres to be crosslinked.

However, protein-based implants treated with glutaraldehyde-type compounds generally lead to rather rapid calcification of the implant when said implant is placed inside a living body.

The calcification results in a hardening of the implant due to the accumulation of calcium salts at the implant. This impairs the properties of the implant, particularly in the use of cardiac or vascular prostheses, which requires periodic replacement of the implant by surgical means. For more details on this subject please refer, in particular, to U.S. Pat. No. 5,645,587.

It has been suggested to post-treat the implants treated with glutaraldehyde with different compounds, in particular with oleic acid. These types of treatment fundamentally aim, in fact, to eliminate the presence of toxic by-products and do not sufficiently limit calcification.

### SUMMARY OF THE INVENTION

An object of the present invention is to provide biocompatible protein-based implants, in which the occurrences of calcification are limited in comparison with those observed with treated protein-based implants currently known, preferably to an extent sufficient to avoid periodic replacement of the implant.

Therefore, according to a first feature, the invention relates to a method for treating an implant comprising a protein-based substrate, said method including the following steps in which:

(A)—the protein-based substrate is treated with a compound containing at least one aldehyde group, preferably with a compound containing at least two aldehyde groups, then

(B)—the substrate is treated with a compound comprising a borohydride, then

(C)—the substrate resulting from step (B) is treated with a derivative containing a silane group.

Within the sense of the present description, “implant” means a device to be implanted inside a living body comprising or being composed of a protein-based substrate. Typically, the treated implant according to the invention is a cardiac implant, in particular a cardiac valve implant.

In this context, “protein-based substrate” means a substrate comprising one or more proteins, generally as a major component, for example at a content between 50 and 100% by weight. Said protein-based substrate constitutes the implant either entirely or in part. More often, the implant is entirely constituted by said protein-based substrate.

The substrate may also cover other materials, such as prostheses, tubes, and surgical equipment in contact with the living environment.

In the method of the invention, the protein-based substrate of the implant is subjected to a modification treatment which ensures, in particular, its biocompatibility. In this context, the term “biocompatibility” of the implant means that when the implant is placed inside a living body, and in particular inside a human body, said implant is not recognised by the immune system which makes it possible to avoid protein-based implants being rejected.

The inventors have now proved that the succession of steps (A), (B) and (C) allows an increased level of biocompatibility to be conferred to a protein-based implant whilst also limiting the occurrence of calcification of said implant.

In particular, inventors' studies have made it possible to establish that the combination of steps (B) and (C) makes it possible to strongly reduce the occurrences of calcification which are observed with implants currently known, which correspond to implants treated solely according to step (A) of the present invention.

The limitation of the occurrence of calcification seems, in part, to be explained by the fact that the succession of steps (B) and (C) allows the number of free aldehyde functions introduced in step (A) to be reduced to alcohol functions (in step (B)), said alcohol functions being protected in the form of siloxane functions (in step (C)). Said siloxane functions have the advantage of being stable functions which are biocompatible and not very conducive to the accumulation of calcium salts.

Also, it has further been proven that the reduction step (B) leads, in addition to the effect mentioned above, to a reduction of other functions introduced in step (A). More precisely, step (B) leads to the reduction of imine functions resulting from the coupling of free amines of the substrate of the implant to the difunctional aldehydes of step (A). This reaction of the imine functions is particularly advantageous since it has been proven that said imine functions also aid calcification. Step (B) thus makes it possible to delete imine groups capable of inducing calcification by converting them into substituted amine functions which have the advantage of being stable.

Thus, the method of the invention makes it possible to limit the occurrence of calcification by inhibiting the presence of two sources responsible for calcification of the implant. Consequently, the implant prepared according to the method of the invention has the advantage of having a low rate of calcification, which makes it possible, in certain cases, to avoid replacing the implant by means of a surgical procedure, or at least to stagger the timing of surgical procedures necessary to replace the implant.

More often, the protein-based substrate present in the implant treated according to the invention comprises or is composed of fibrous proteins. Preferably, the substrate is collagen-based, elastin-based, fibrin-based, fibrinogen-based and/or proteoglycan-based.

The implant treated according to the invention is typically a cardiac valve implant including all or part of a bovine, porcine, ovine, equine or ostrich aortic valve and/or pericardium.

Considering the presence of proteins, the substrate of the implant inherently comprises free amine functions which would be responsible, at least in part, for rejection if the untreated substrate were implanted into a living body without being treated.

In step (A) of the method according to the invention, the protein-based substrate constituting the implant is treated with a compound containing at least one aldehyde group.

For the sake of conciseness, said compound will be referred to hereinafter by the generic term “aldehyde compound”. The aldehyde group of step (A) is likely to react upon the free amine functions of the substrate by transforming said functions into imine functions. Generally, the compound containing at least one aldehyde function is a compound represented by the following formula (I):

R—CHO (formula I)

where R is a hydrocarbon chain typically comprising between 2 and 18 carbon atoms, for example between 3 and 8 carbon atoms optionally substituted with a heteroatom, such as a chlorine, fluorine, bromine, nitrogen, phosphorous or sulphur atom. Said group R may optionally be substituted with one or more other aldehyde —CHO groups.

Advantageously, the aldehyde compound of step (A) is water-soluble.

### DESCRIPTION OF PREFERRED EMBODIMENTS

According to an advantageous embodiment, the aldehyde compound used in step (A) is a compound containing at least two aldehyde —CHO groups, which makes it possible for some protein fibres of the substrate of the implant treated according to the method of the invention to be crosslinked. In this context, the aldehyde compound of step (A) may, in particular, be of general formula (II): HOC—R²—CHO where R² is a hydrocarbon chain typically comprising between 2 and 18 carbon atoms, for example between 3 and 8 carbon atoms optionally substituted by a heteroatom, such as a chlorine, fluorine, bromine, nitrogen, phosphorous or sulphur atom. Preferably, the aldehyde compound of step (A) is glutaraldehyde.

According to a significant embodiment of step (A), the substrate of the implant is immersed in a solution (S_{A}), in particular an aqueous solution, containing the aldehyde compound for at least 2 weeks, preferably for at least 1 month. The solution (S_{A}) used according to this embodiment may, in particular, be prepared by diluting the aldehyde compound in a buffer, the pH of the solution (S_{A}) being, preferably, between 5 and 9. By way of example, when the aldehyde compound is glutaraldehyde, the pH of the solution (S_{A}) is approximately between 6 and 8. In particular, an aqueous solution of sodium phosphate, potassium phosphate or HEPES (4-(2-hydroxyethyl)-1-piperazine ethane sulfonic acid) may be used as a buffer at a concentration of, preferably, between 10 and 50 mmol.l⁻¹, for example approximately 20 mmol.l⁻¹. The concentration of aldehyde compound in the solution (S_{A}) is typically between 0.1 and 1%, for example approximately 0.6 to 0.7% by weight relative to the total volume of the solution (S_{A}). In addition, a solution (S_{A}) containing the aldehyde compound with an osmolarity (corresponding to the number of moles of solute per kilogram of solvent) between 200 and 400 mOsMol.l⁻¹, for example approximately 300 mOsMol.l⁻¹, is preferably used.

The treatment of step (A) is preferably carried out with stirring. In addition, the temperature of the reaction medium of step (A) is, preferably, between 10 and 70° C. For example, step (A) may be carried out at ambient temperature, typically between 20 and 30° C., in particular at approximately 25° C.

More often, the treatment of step (A) leads to a reaction between the free amine functions and the aldehyde compound, according to the following schematic reaction.

[substrate]-NH₂+R—CHO→[substrate]-N═CH—R

where R has the meaning defined above.

More often and in particular, when the preferred solutions mentioned above are used, step (A) leads to a reaction of most and even all of the free amine functions initially present on the substrate of the implant.

In the case of using a compound containing at least two aldehyde functions, step (A) further leads to reactions which induce crosslinking between some fibres of the substrate, according to the following schematic reaction:

2[substrate]-NH₂+OHC—R²—CHO→[substrate]-N═CH—R²—CH═N-[substrate]

where R² has the meaning defined above.

With regard to difunctional aldehyde compounds, one of the aldehyde functions of said aldehyde compounds may remain free in some cases according to the following reaction:

[substrate]-NH₂+OHC—R²—CHO-[substrate]-N═CH—R²—CHO

where R² has the meaning defined above.

Thus, at the end of step (A), free aldehyde —CHO groups remain which are likely to induce calcification. Moreover, the substrate contains imine functions (—N═C—) which are also sources of calcification.

It is this type of reaction which is observed when collagen-based implants are treated with glutaraldehyde according to the methods known from the state of the art.

An object of steps (B) and (C) is to delete substantially all said free aldehyde —CHO groups and the imine functions introduced at the end of step (A).

In step (B) of the method according to the invention, the substrate is treated with a compound comprising a borohydride, which allows aldehyde functions to be reduced to alcohol functions and also for imine functions to be reduced to amine functions in such a selective manner that the amide functions constituting the proteins are not modified.

The compound comprising a borohydride which is used in step (B) is preferably an alkali metal derivative, such as sodium, lithium or potassium. Preferably, the compound comprising a borohydride is a metal cyanoborohydride, such as sodium cyanoborohydride.

According to a beneficial embodiment, step (B) may be carried out by partially or totally immersing the substrate resulting from step (A) of the method according to the invention in a solution (S_{B}) of the compound comprising a borohydride, typically for at least 1 hour, generally for at least 5 hours, for example for between 10 and 30 hours, typically for approximately 24 hours. The solution (S_{B}) used according to the embodiment may, in particular, be obtained by diluting the compound comprising a borohydride in a buffer solution. The solution (S_{B}) typically has a pH between 5 and 11. A suitable buffer is, in particular, an aqueous solution comprising sodium disodium phosphate, of which the concentration is typically between 20 and 500 mmol.l⁻¹, for example approximately 200 mmol.l⁻¹. The concentration of the compound comprising a borohydride is, in particular, between 10 and 160 mmol.l⁻¹, preferably equal to approximately 80 mmol.l⁻¹.

The treatment of step (B) is generally carried out with stirring, for example at approximately 50 rpm⁻¹. The temperature of the reaction medium of step (B) is, preferably, between 10 and 70° C. For example, step (B) may be carried out at ambient temperature, for example between 20 and 30° C., typically at approximately 25° C.

Typically, the reaction which takes place in step (B) is the following:

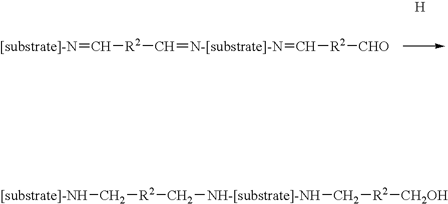

where R² has the meaning defined above.

Thus, at the end of step (B), the substrate comprises terminal alcohol functions capable of reacting with the phosphorylation enzymes, which is, in particular, likely to provoke degradation of the implant. In fact, the phosphate groups of said enzymes link easily to calcium cations by initiating calcification of the substrate. Eventually, such degradation of the implant would also require replacement of said implant by surgical means.

An object of step (C) is to eradicate the presence of terminal alcohol functions introduced in step (B). For this purpose, in step (C) of the method of the invention, the substrate resulting from step (B) is treated with a derivative containing a silane group so as to convert the alcohol functions into siloxane functions. The siloxane functions thus formed are unreactive and are also definitive in the sense that the reaction for protecting alcohol functions into siloxane functions is irreversible (deprotection would involve destruction of the substrate). This definitive protection of the terminal alcohol functions means it is possible to avoid any degradation of the implant by active compounds issued from a living organism. Moreover, the siloxane functions are not recognised by the immune system and do not aid calcification.

Generally speaking, the derivative containing a silane group used in step (C) comprises an electroattractive group linked directly to the silicon atom. Said electroattractive group is typically selected from the halogens, the heteroaryl groups comprising between 5 and 15 carbon atoms and, optionally, 2 or 3 heteroatoms typically selected from the group consisting of the halogens, such as fluorine, chlorine, bromine and iodine, the pnictogens corresponding to the elements of the fifth column of the periodic table of the elements, such as nitrogen and phosphorous, and the chalcogens corresponding to the elements in the sixteenth column of the periodic table, such as oxygen and sulphur.

Preferably, the electroattractive group present in the derivative containing a silane group used in step (C) is a chlorine atom, a bromine atom or an imidazole group.

According to a variation, the electroattractive group may be those mentioned above known by the person skilled in the art.

Preferably, the derivative containing a silane group used in step (C) is a trialkylsilylimidazole or a halogenotrialkylsilane, for example trimethylsilylimidazole or chlorotrimethylsilane (also called chloromethylsilane hereafter).

Preferably, the derivative containing a silane group used in step (C) is introduced in the form of a solution (S_{C}) in which at least one water-soluble non-toxic compound selected from the group consisting of tetrahydrofuran or even dioxane, diglyme, triglyme, dimethylformamide, dimethylacetamide, or N-methylpyrrolidone is used as solvent. By way of example, the derivative containing a silane group may be diluted in the tetrahydrofuran according to a dilution factor between 1 and 20%, preferably equal to approximately 10%.

Typically, in step (C), the substrate resulting from step (B) is treated in the solution (S_{C}) mentioned above for between 1 and 30 minutes, for example between 4 and 6 minutes, typically for approximately 5 min. The treatment in step (C) may be carried out, in particular, with stirring, for example at approximately 50 rpm⁻¹. In addition, in step (C), the temperature of the reaction medium is preferably between 10 and 35° C. For example, step (C) may be carried out at ambient temperature, for example between 20 and 30° C., typically at approximately 25° C.

According to an advantageous variation, the method according to the invention comprises, in addition to the aforementioned steps (A), (B) and (C), an intermediate step (A1) arranged between steps (A) and (B), in which the substrate as obtained at the end of step (A) is treated with a compound containing at least two amine functions before steps (B) and (C) are carried out.

According to this variation of the method of the invention, part of the free aldehyde functions formed at the end of step (A) react upon the amine functions present on the compounds containing amine functions used in step (A1), thus forming imine links according to the following schematic reaction:

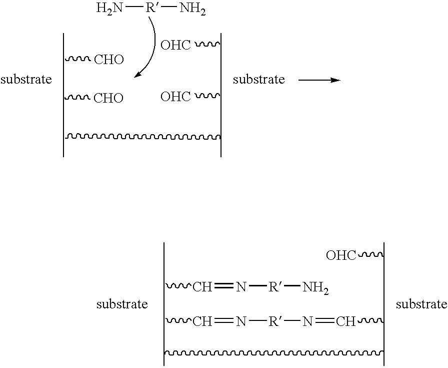

As the diagram above illustrates, step (A1) leads, among other advantages, to an increase in crosslinking between the protein chains of the substrate.

In addition, step (A1) masks part of the aldehyde —CHO functions remaining free at the end of step (A). As shown in the drawing, this masking may induce the formation of grafted chains containing free terminal amine functions. The presence of said free terminal amine functions is, however, not detrimental to the biocompatibility of the implant. In fact, said functions are not recognised by the immune system and therefore do not lead to rejection.

Preferably, the compound containing at least two amine functions used in step (A1) is a diamine of formula NH₂—A—NH₂ where A represents a linear or branched hydrocarbon chain comprising between 1 and 20 carbon atoms optionally substituted with one or more heteroatoms selected from the group consisting of the halogens, such as fluorine, chlorine, bromine and iodine, the pnictogens corresponding to the elements in column Vb of the periodic table of elements, such as nitrogen and phosphorous, and the chalcogens corresponding to the elements in column Vlb of the periodic table, such as oxygen and sulphur. Even more preferably, the compound containing at least two amine function is poly(propylene glycol)bis(2-aminopropyl ether), lysine, spermine or putrescine.

According to one embodiment, step (A1) may be carried out by partially or totally immersing the implant resulting from step (A) of the method according to the invention in an aqueous solution (S_{A1}) containing the compound containing at least one amine function, typically at a concentration between 10 and 200 mmol.l⁻¹, preferably between 40 and 80 mmol.l⁻¹, for example at a concentration of approximately 60 mmol.l⁻¹. Typically, step (A1) is carried out by partially or totally immersing the implant in the solution (S_{A1}) typically for between 10 and 100 min, for example for approximately 60 min.

The treatment in step (A1) may typically be carried out with stirring, for example at approximately 50 rpm⁻¹. The temperature of the reaction medium of step (A1) is preferably between 10 and 70° C. For example, step (A1) may be carried out at ambient temperature, for example between 20 and 30° C., typically at approximately 25° C.

According to a particular embodiment, following step (A) and optionally following step (A1) and before step (B), the method of the invention may further include a step for modifying pH, so as to bring the reaction medium to a pH, in particular, between 5 and 9, preferably between 5.5 and 6.5, for example equal to approximately 6, which aids the subsequent reduction of step (B). This step may be carried out, in particular, by treating the medium resulting from step (A) and resulting from the optional step (A1) with morpholinoethanesulfonic acid (MES) for between 10 and 50 hours, preferably between 20 and 30 hours, for example for approximately 24 hours.

According to one embodiment, before and after each step of a method of the invention, the implant may be washed with water, typically with ultra pure water. In this context, “ultra pure water” means that the resistance of the water is equal to approximately 18.2 MΩ.cm at approximately 25° C. Washing with water, in particular with ultra pure water, eliminates the excess reactants present at the end of each of the steps of the method, which renders the implant free from any compound which could interact with the organism of the living body. By way of example, the implant may be rinsed at least two times, preferably three times, with ultra pure water before and after each of steps (A), (B) and (C) and, optionally, (A1).

According to one embodiment, the medium of step (A) may be isolated so as to conserve the implant for the subsequent implementation of steps (B) and (C) or of steps (A1), (B) and (C).

According to one embodiment, the implant treated according to steps (A), optionally (A1), (B) and (C) may be subjected to another treatment following step (C). Said additional treatment may be carried out so as to improve even further the resistance of the implant to calcification. By way of example, the implant resulting from step (C) may be treated with anticalcifying solutions currently known, such as the “sterilant” (22% ethanol, 4% formaldehyde and 1.2% Tween 80 (polysorbate 80)) in the rest of the water, the percentages being given by volume relative to the total volume of the solution.

Whatever the method of carrying out the method of the invention, at the end of said method an implant is obtained which no longer substantially contains functions capable of inducing calcification.

According to a second feature, the invention also relates to a treated protein-based implant which is likely to be obtained at the end of the method of the invention.

A treated implant according to the invention is generally substantially free of free aldehyde —CHO functions and imine functions.

In addition, the implant treated according to the invention contains terminal siloxane functions.

Terminal siloxane functions means hydrocarbon chains comprising heteroatoms, such as a nitrogen, oxygen, chlorine, bromine, iodine or phosphorous atom interrupted with a silicon atom on the surface of the implant.

Thus, the implant according to the invention has the advantage of having low calcification.

Preferably, the implant treated according to the invention is a cardiac valve implant.

Different features and advantages of the invention will be revealed upon reading the following non-limiting examples.

### EXAMPLES

Substrate Used

To prepare the implant, part of a bovine pericardium was used as a substrate.

Preparation of the Solutions

In the following examples, the ultra pure water used is an aqueous solution having a resistance equal to approximately 18.2 MΩ at approximately 25° C.

Glutaraldehyde Solution (S1)

Approximately 6.25 g of glutaraldehyde were diluted in approximately 1 l of a buffer solution comprising sodium phosphate and potassium phosphate at approximately 20 mmol.l⁻¹. A final concentration of glutaraldehyde was thus obtained of approximately 0.625% by weight relative to the total volume of the solution (S1).

The osmolarity of the solution was equal to approximately 300 mOsmol.l⁻¹ by adding approximately 5.3 g of sodium chloride to the solution (S1).

The pH of the solution (S1) was approximately 7.4.

Poly(propylene glycol)bis(2-aminopropyl ether) (Jeffamine) solution (S2)

Approximately 1.437 ml of poly(propylene glycol)bis(2-aminopropyl ether) were diluted in approximately 98.563 ml of ultra pure water.

The concentration of poly(propylene glycol)bis(2-aminopropyl ether) in the solution (S2) was equal to approximately 60 mmol.l⁻¹.

Sodium Cyanoborohydride (NaCNBH₃) Solution (S3)

Approximately 0.5 g of sodium cyanoborohydride was dissolved in approximately 10 ml of ultra pure water for one night. The solution obtained was then diluted in approximately 90 ml of another solution comprising 200 mmol.l⁻¹ of Na₂HPO₄.

The final concentration of sodium cyanoborohydride in the solution (S3) was approximately 80 mmol.l⁻¹.

Chloromethylsilane(chorotrimethylsilane)solution (S4)

Approximately 10 ml of chloromethylsilane were diluted in approximately 90 ml of tetrahydrofuran.

Trimethylsilylimidazole Solution (S5)

Approximately 10 ml of trimethylsilylimidazole were diluted in approximately 90 ml of tetrahydrofuran.

“Sterilant” Solution (S6)

Approximately 220 ml of absolute ethanol, 108 ml of formaldehyde at 37% and 12 ml of Tween 80 were diluted in approximately 660 ml of sodium phosphate and potassium phosphate buffer. The final concentration of phosphate was approximately 20 mmol.l⁻¹ and the pH of the solution was approximately 7.4.

### Example 1

### Treatment of Substrate According to Steps (A) (B) and (C) of the Method of the Invention

Treatment with Glutaraldehyde Solution (S1)

The substrate was treated with solution (S1) for at least one month at ambient temperature (approximately 25° C.).

At the end of said treatment, the treated substrate was cut into squares measuring 8 mm, approximately 7 mm, on each side.

The samples resulting from the substrate thus treated were rinsed three times with ultra pure water.

Treatment with Sodium Cyanoborohydride Solution (S3)

Said rinsed samples were transferred to a 150 ml bottle with a rectangular cross-section containing approximately 100 ml of solution (S3). The reaction medium was then stirred at approximately 50 rpm⁻¹ at ambient temperature (approximately 25° C.) for approximately 24 hours.

The samples thus treated were rinsed three times with ultra pure water.

Treatment with Chloromethylsilane Solution (S4)

The samples thus rinsed were transferred to a 150 ml bottle with a rectangular cross-section containing 100 ml of the chloromethylsilane solution (S4). The reaction medium was stirred at approximately 50 rpm⁻¹ at ambient temperature (approximately 25° C.) for approximately 5 minutes.

The samples thus treated were rinsed three times with ultra pure water.

### Example 2

### Treatment of the Substrate According to Steps (A), (A1), (B) and (C) of the Method of the Invention

Treatment with Glutaraldehyde Solution (S1)

The substrate was treated with solution (S1) for at least one month at ambient temperature (approximately 25° C.).

At the end of said treatment, the treated substrate was cut into squares measuring approximately 7 mm on each side.

The samples resulting from the substrate thus treated were rinsed three times with ultra pure water.

Treatment with poly(propylene glycol)bis(2-aminopropyl ether) (Jeffamines) Solution (S2)

The samples thus rinsed were transferred to a 150 ml bottle with a rectangular cross-section containing 100 ml of solution (S2). The bottle was stirred at approximately 50 rpm⁻¹ for approximately 1 hour at ambient temperature (approximately 25° C.).

Approximately 5.76 g of morpholinoethanesulfonic acid (MES) were added to the reaction medium. The reaction medium was stirred at approximately 50 rpm⁻¹ at ambient temperature (approximately 25° C.) for approximately 23 hours.

The final pH of the reaction medium was approximately 6.

The samples thus treated were rinsed three times with ultra pure water.

Treatment with Sodium Cyanoborohydride Solution (S3)

Said rinsed samples were transferred to a 150 ml bottle with a rectangular cross-section containing approximately 100 ml of solution (S3). The reaction medium was then stirred at approximately 50 rpm⁻¹ at ambient temperature (approximately 25° C.) for approximately 24 hours.

The samples thus treated were rinsed three times with ultra pure water.

Treatment with Chloromethylsilane Solution (S4)

The samples thus rinsed were transferred to a 150 ml bottle with a rectangular cross-section containing approximately 100 ml of the chloromethylsilane solution (S4). The reaction medium was then stirred at approximately 50 rpm⁻¹ at ambient temperature (approximately 25° C.) for approximately 5 minutes

The samples thus treated were rinsed three times with ultra pure water.

### Example 3

### Treatment of the Substrate According to Steps (A) (A1), (B) and (C) of the Method of the Invention

The same procedure was repeated except that in the last step (C) the trimethylsilylimidazole solution (S5) was used instead of the chloromethylsilane solution (S4).

### Example 4

### Treatment of the Substrate According to Steps (A) (A1), (B) then (C) of the Method of the Invention Followed by a Subsequent Step of Treatment with an Anticalcifying Solution (Sterilant)

The procedure of example 3 was adopted, and then the following step was carried out.

The samples thus treated were transferred to a 150 ml bottle with a rectangular cross-section containing 100 ml of solution (S6). The reaction medium was then stirred at approximately 50 rpm⁻¹ at ambient temperature (approximately 25° C.) for approximately 24 hours.

The samples thus treated were rinsed three times with ultra pure water.

### Comparative Examples

### Comparative Example 1

### Treatment of Substrate According to Step (A) Only

The substrate was treated with the glutaraldehyde solution (S1) for at least one month at ambient temperature (approximately 25° C.).

At the end of said treatment, the treated substrate was cut into squares measuring 7 mm on each side.

The samples resulting from the substrate thus treated were rinsed three times with ultra pure water then were kept in solution (S1) until they were implanted in rats.

### Comparative Example 2

### Treatment of the Substrate According to Step (A) Followed by a Step of Treatment with the Sterilant Solution (S6)

The same procedure as comparative example 1 was adopted, at the end of which the following step was carried out.

The samples thus treated with the glutaraldehyde solution (S1) were transferred to a 150 ml bottle with a rectangular cross-section containing 100 ml of an aqueous solution (S6). The reaction medium was stirred at approximately 50 rpm⁻¹ at approximately 32° C. for approximately 9 hours.

The samples thus treated were rinsed three times with ultra pure water.

### Comparative Example 3

### Treatment of the Substrate According to Step (A) Followed by a Treatment Step with Tetrahydrofuran

The same procedure as comparative example 1 was adopted, at the end of which the following step was carried out.

The samples thus treated with the glutaraldehyde solution (S1) were transferred to a 150 ml bottle with a rectangular cross-section containing 100 ml tetrahydrofuran. The reaction medium was stirred at approximately 50 rpm⁻¹ at ambient temperature (approximately 25° C.) for approximately 24 hours.

The samples thus treated were rinsed three times with ultra pure water.

### Comparative Example 4

### Treatment of the Substrate According to Step (A) Followed by Step (C) using Solution (S4)

The same procedure as comparative example 1 was adopted, at the end of which the following step was carried out.

The samples thus treated with the glutaraldehyde solution (S1) were transferred to a 150 ml bottle with a rectangular cross-section containing 100 ml of the chloromethylsilane solution (S4). The reaction medium was then stirred at approximately 50 rpm⁻¹ at ambient temperature (approximately 25° C.) for approximately 5 minutes.

The samples thus treated were rinsed three times with ultra pure water.

### Comparative Example 5

### Treatment of the Substrate According to Step (A) Followed by Step (C) using Solution (S5)

The procedure of comparative example 4 was adopted, replacing the chloromethylsilane solution (S4) with the trimethylsilylimidazole solution (S5).

### Comparative Example 6

### Treatment of the Substrate According to Steps (A) then (C) using Solution (S5) Followed by a Step of Treatment using the Sterilant Solution (S6)

The procedure of comparative example 5 was adopted, at the end of which the following step was carried out.

The samples thus treated with the glutaraldehyde solution (S1) were transferred to a 150 ml bottle with a rectangular cross-section containing 100 ml of solution (S6). The reaction medium was then stirred at approximately 50 rpm⁻¹ at ambient temperature (approximately 25° C.) for approximately 24 hours.

The samples thus treated were rinsed three times with ultra pure water.

### Comparative Example 7

### Treatment of the Substrate According to Steps (A), (A1) then (C) using Solution (S4)

The same procedure as comparative example 1 was adopted, at the end of which the following steps were carried out.

Treatment with the poly(propylene glycol)bis(2-aminopropyl ether)solution (S2)

The samples thus treated with solution were transferred to a 150 ml bottle with a rectangular cross-section containing 100 ml of solution (S2). The bottle was stirred at approximately 50 rpm⁻¹ for approximately 1 hour at ambient temperature (approximately 25° C.).

Approximately 5.76 g of morpholinoethanesulfonic acid (MES) were added to the reaction medium. The reaction medium was stirred at approximately 50 rpm⁻¹ at ambient temperature (approximately 25° C.) for approximately 23 hours.

The final pH of the reaction medium was approximately 6.

The samples thus treated were rinsed three times with ultra pure water.

Treatment with the Chloromethylsilane Solution (S4)

The samples thus rinsed were transferred to a 150 ml bottle with a rectangular cross-section containing 100 ml of the chloromethylsilane solution (S4). The reaction medium was then stirred at approximately 50 rpm⁻¹ at ambient temperature (approximately 25° C.) for approximately 5 minutes.

The samples thus treated were rinsed three times with ultra pure water.

Study of Calcification in Rats

The untreated or treated samples resulting from the examples described above were implanted subcutaneously in newborn rats aged 12 days.

The rats were weaned 9 days after implantation and were fed a diet consisting of a portion of grains containing approximately 332 mg calcium, approximately 236 mg phosphorous, approximately 9.6 mg iron, approximately 60 UI vitamin D3 per kg of rat and unlimited water.

Ten months after implantation, the rats were killed and the samples were explanted so as to be analysed.

The samples were cleaned using ultra pure water, lyophilised then weighed (dry weight in mg). The lyophilised samples were digested in approximately 1 ml of 70% nitric acid at approximately 95° C. for approximately 15 min. The volume of the medium was then made up to approximately 5 ml with ultra pure water in a 5 ml volumetric flask.

The calcium of said samples was assayed using a flame atomic spectrophotometer. The calcium thus assayed originates essentially from calcification of the implant.

The results of the calcium assay obtained from the samples having been subjected or not to different treatments are shown in the following table.

| | Percentage of calcium relative to |
|---|---|
| Disc treated with | total weight of disc |
| No treatment | 9.75% |
| Example 1 | 0.67% |
| Example 2 | 0.07% |
| Example 3 | 0.56% |
| Example 4 | 0.49% |
| Comparative example 1 | 20.82% |
| Comparative example 2 | 1.67% |
| Comparative example 3 | 9.10% |
| Comparative example 4 | 1.09% |
| Comparative example 5 | 1.15% |
| Comparative example 6 | 1.33% |
| Comparative example 7 | 1.14% |

Glut. = glutaraldehyde

According to the results shown in the table above, the substrate treated with sodium cyanoborohydride followed by treatment with a derivative containing a silane group, in particular with chloromethylsilane or trimethylsilylimidazole, clearly reduces calcification in comparison with treatment using the commercial solution, the sterilant.

Furthermore, if the implant is further treated with a compound containing at least two amine functions, poly(propylene glycol)bis(2-aminopropyl ether), calcification is reduced even further.

1. Method for treating an implant comprising a protein-based substrate, including the following steps, in which:
(A)—the protein-based substrate is treated with a compound containing at least one aldehyde group, then
(B)—the substrate is treated with a compound comprising a borohydride, then
(C)—the substrate resulting from step (B) is treated with a derivative containing a silane group.

2. Method according to claim 1, wherein the protein-based substrate is collagen-based, elastin-based, fibrin-based, fibrinogen-based and/or proteoglycan-based.

3. Method according to claim 1, wherein the implant is a cardiac valve implant, including all or part of a bovine, porcine or ovine aortic valve and/or pericardium.

4. Method according to claim 1, wherein in step (A) a compound containing at least two aldehyde groups is used.

5. Method according to claim 1, wherein the compound comprising a borohydride which is used in step (B) is an alkali metal derivative.

6. Method according to claim 5, wherein the compound comprising a borohydride which is used in step (B) is sodium cyanoborohydride.

7. Method according to claim 1, wherein the derivative containing a silane group used in step (C) comprises an electroattractive group linked directly to the silicon atom and selected from the halogens, the heteroaryl groups comprising between 5 and 15 carbon atoms and 2 or 3 heteroatoms selected from the group consisting of the halogens, pnictogens and chalcogens.

8. Method according to claim 7, wherein the electroattractive group present in the derivative containing a silane group used in step (C) is a chlorine atom, a bromine atom or an imidazole group.

9. Method according to claim 8, wherein the derivative containing a silane group used in step (C) is trimethylsilylimidazole or chlorotrimethylsilane.

10. Method according to claim 1, including an intermediate step (A1) between steps (A) and (B), wherein the substrate obtained at the end of step (A) is treated with a compound containing at least two amine functions before steps (B) and (C) are carried out.

11. Method according to claim 10, wherein the compound containing at least two amine functions is a diamine of formula NH₂-A-NH₂ where A represents a linear or branched hydrocarbon chain comprising between 1 and 20 carbon atoms optionally substituted with one or more heteroatoms selected from the group consisting of the halogens, pnictogens and chalcogens.

12. Method according to claim 11, wherein the compound containing at least two amine functions is poly(propylene glycol)bis(2-aminopropyl ether), lysine, spermine or putrescine.

13. Treated protein-based implant which is likely to be obtained at the end of the treatment method according to claim 1.

14. Implant according to claim 13, wherein said implant is substantially free of free aldehyde —CHO functions and imine functions.

15. Implant according to either claim 13, wherein said implant is a cardiac valve implant.
